(19) United States
(12) Patent Application Publication
(10) Pub. No.: US 2021/0085156 A1
(43) Pub. Date: Mar. 25, 2021

(54) RIGIDITY VARIABLE DEVICE AND ENDOSCOPE

(71) Applicant: OLYMPUS CORPORATION, Tokyo (JP)

(72) Inventor: Tomoyuki KUGO, Matsudo-shi (JP)

(73) Assignee: OLYMPUS CORPORATION, Tokyo (JP)

(21) Appl. No.: 17/109,251

(22) Filed: Dec. 2, 2020

Related U.S. Application Data
(63) Continuation of application No. PCT/JP2018/021382, filed on Jun. 4, 2018.

(51) Int. Cl. A61B 1/005

(52) U.S. Cl. CPC A61B 1/0058; A61B 1/0051

(57) ABSTRACT

A rigidity variable device includes: an elongated main body unit; a plurality of metal high-rigidity portions arranged with gaps along an longitudinal axis of the main body unit in the main body unit, the plurality of high-rigidity portions being made of metal; and a low-rigidity portion including a plurality of shape-memory alloy wires that intervene between high-rigidity portions adjacent to each other among the high-rigidity portions, and are apart in a direction orthogonal to the longitudinal axis in the main body unit.

### CROSS REFERENCE TO RELATED APPLICATION

This application is a continuation application of PCT/JP2018/021382 filed on Jun. 4, 2018, the entire contents of which are incorporated herein by this reference.

### BACKGROUND OF THE INVENTION

### 1. Field of the Invention

The present invention relates to a rigidity variable device that adopts a shape-memory alloy, and an endoscope.

### 2. Description of the Related Art

For example, as disclosed in International Publication No. WO 2016/174741, a scheme has been proposed that provides an elongated shape-memory member and provides a heating coil separately from the shape-memory alloy, and improves the rigidity by heating the shape-memory member through the heating coil, as a rigidity variable device that adopts a shape-memory alloy.

### SUMMARY OF THE INVENTION

A rigidity variable device according to an aspect of the present invention includes: an elongated main body unit; a plurality of high-rigidity portions arranged with gaps along an longitudinal axis of the main body unit in the main body unit, the plurality of high-rigidity portions being made of metal; and a low-rigidity portion including a plurality of shape-memory alloy wires that intervene between high-rigidity portions adjacent to each other among the high-rigidity portions, and are apart in a direction orthogonal to the longitudinal axis in the main body unit.

An endoscope according to an aspect of the present invention includes: an insertion portion configured to be inserted into a subject; and the rigidity variable device.

### BRIEF DESCRIPTION OF THE DRAWINGS

FIG. 1 is a diagram showing a configuration of a rigidity variable device;

FIG. 2 is a sectional view taken along line II-II of FIG. 1; and

FIG. 3 is a diagram showing a configuration of an endoscope.

### DETAILED DESCRIPTION OF THE PREFERRED EMBODIMENT

A preferable mode of the present invention is hereinafter described with reference to the drawings. In each diagram used in the following description, each component is caused to have a different scale on a component-by-component basis so as to achieve a size to an extent recognizable on the diagram. The present invention is not limited only to the number of components, the shapes of the components, the ratios of the sizes of the components, and relative positional relationships between components that are described in these diagrams.

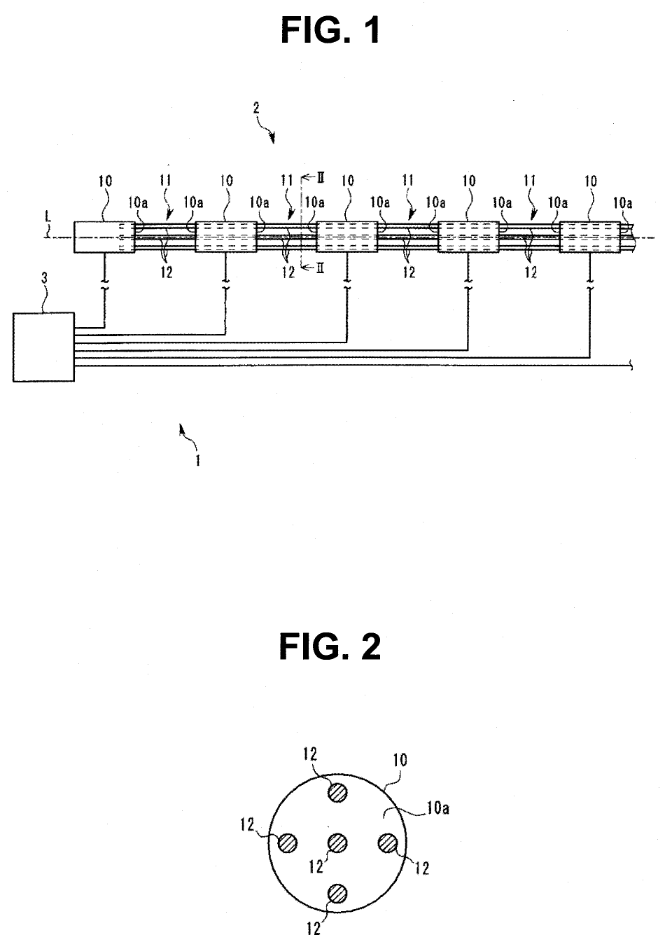

An example of an embodiment of the present invention is hereinafter described. A rigidity variable device 1 shown in FIG. 1 includes a main body unit 2, and an energization member 3. The main body unit 2 has an elongated shape along a longitudinal axis L. The rigidity variable device 1 can change the rigidity in response to an input of a force in a direction of bending the longitudinal axis L of the main body unit 2. Here, the rigidity indicates difficulty of bending deformation of the elongated main body unit 2. The rigidity is represented by a force required to bend an interval having a predetermined length, to a predetermined curvature, in a direction along the longitudinal axis L of the main body unit 2. Consequently, the higher the rigidity is, the more resistant to the deformation in the bending direction the main body unit 2 is.

The main body unit 2 includes a plurality of high-rigidity portions 10 and low-rigidity portions 11. The plurality of high-rigidity portions 10 are arranged in a single row along the longitudinal axis L. Parts of an adjacent pair of high-rigidity portions 10 that face each other are called ends 10a.

A gap is provided between the ends 10a of each adjacent pair of high-rigidity portions 10. The low-rigidity portion 11 is arranged in the gap provided between each adjacent pair of high-rigidity portions 10.

In other words, the ends 10a of each pair of high-rigidity portions 10 are arranged on the opposite sides of the corresponding low-rigidity portion 11 in the direction along the longitudinal axis L. Each low-rigidity portion 11 is fixed to both the ends 10a of the adjacent pair of high-rigidity portions 10. Consequently, the main body unit 2 includes the high-rigidity portions 10 and the low-rigidity portions 11 alternately connected in the direction along the longitudinal axis L.

Note that the numbers of the high-rigidity portions 10 and the low-rigidity portions 11 included in the main body unit 2 are not specifically limited. In this embodiment, for example, five high-rigidity portions 10 and four low-rigidity portions 11 are shown in FIG. 1. The numbers of the high-rigidity portions 10 and the low-rigidity portions 11 may be greater or less than the numbers in this embodiment shown in FIG. 1.

The terms “high rigidity” and “low-rigidity” of the names of the high-rigidity portions 10 and the low-rigidity portions 11, which are described later in detail, are used to represent a relative difference in rigidity between both the components. Consequently, the absolute values of the rigidities of the high-rigidity portions 10 and the low-rigidity portions 11 are not limited by these terms.

The high-rigidity portions 10 are made from metal. Although the shapes of the high-rigidity portions 10 are not specifically limited, the high-rigidity portions 10 are columnar In this embodiment, for example, each high-rigidity portion 10 is cylindrical, and is arranged in a manner allowing the portion to be a circle when viewed in the direction along the longitudinal axis L.

Each low-rigidity portion 11 includes a plurality of shape-memory alloy wires (hereinafter, called SMA wires) 12. Each SMA wire 12 is a linear member made of a shape-memory alloy. The shape memorized by the SMA wire 12 is a linear shape. The shape-memory alloy, which is not described in detail because the shape-memory alloy is of a publicly known technology, causes a phase change at a predetermined temperature T as a boundary, and changes the elastic modulus. The SMA wires 12 in this embodiment cause a phase change at the predetermined temperature exceeding a room temperature, and has a higher elastic modulus at a temperature equal to or higher than the predetermined temperature T than the elastic modulus at a temperature lower than the predetermined temperature T. The SMA wires 12 exhibits superelasticity at temperatures equal to or higher than the predetermined temperature T.

The plurality of SMA wires 12 are electrically connected to an energization member 3 described later. The plurality of SMA wires 12 produce heat to a temperature exceeding the predetermined temperature T, at which a phase change occurs, by heating through energization.

The plurality of SMA wires 12 are arranged between the ends 10a of the pair of high-rigidity portions 10 that are adjacent in a state of being separated from each other. Each of the SMA wires 12 is fixed to both the pair of high-rigidity portions 10.

The method of fixing the SMA wires 12 and the high-rigidity portions 10 is not specifically limited. In this embodiment, for example, the SMA wires 12 and the high-rigidity portions 10 are fixed to each other with conductive adhesive. Note that fixation between the SMA wires 12 and the high-rigidity portions 10 may be made by swaging or soldering, for example.

When the temperature is equal to or higher than the predetermined temperature T and each of the SMA wires 12 has a linear shape, the SMA wire 12 is arranged such that the longitudinal direction is substantially parallel with the longitudinal axis L of the main body unit 2. All the SMA wires 12 included in the low-rigidity portions 11 are narrower than the high-rigidity portions 10. When the temperature is equal to or higher than the predetermined temperature T and the SMA wires 12 have a linear shape, the SMA wires 12 are arranged so as to be apart from each other. In other words, the plurality of SMA wires 12 are arranged so as to be apart from each other in directions orthogonal to the longitudinal axis L.

The number of SMA wires 12 included in each low-rigidity portion 11 is not specifically limited, only if the number is two or more. In this embodiment, for example, the low-rigidity portions 11 include five SMA wires 12 as shown in FIG. 2.

The arrangement of the plurality of SMA wires 12 is not specifically limited. In this embodiment, for example, on a section orthogonal to the longitudinal axis L, one SMA wire 12 is arranged on the central axis of the cylindrical high-rigidity portion 10, and the remaining four SMA wires 12 are arranged around the central axis at a regular interval (90 degrees) in the circumferential direction.

The plurality of low-rigidity portions 11 included in the main body unit 2 may include SMA wires 12 that are different from each other, or share the same SMA wires 12. For example, in this embodiment, each of the low-rigidity portions 11 may independently include five SMA wires 12.

Alternatively, for example, at least two low-rigidity portions 11 among the low-rigidity portions 11 may be made up of common five SMA wires 12. In this case, the five SMA wires 12 penetrate through the high-rigidity portion 10 intervening between the two low-rigidity portions 11.

In this embodiment, for example, all the low-rigidity portions 11 included in the main body unit 2 are made up of common five SMA wires 12. In other words, in the main body unit 2 of this embodiment, the five SMA wires 12 extend in parallel with the longitudinal axis L and are arranged apart from each other in directions orthogonal to the longitudinal axis L, and the plurality of high-rigidity portions 10 are fixed by the five SMA wires 12 in a state where the high-rigidity portions 10 are apart from each other in the direction along the longitudinal axis L.

As described above, the SMA wires 12 in this embodiment are fixed to the metal high-rigidity portions 10 with conductive adhesive. Accordingly, the five SMA wires 12 are electrically connected to each other through the high-rigidity portions 10.

The energization member 3 switches presence or absence of energization to the SMA wires 12. Note that the energization member 3 is only required to have a function of switching presence or absence of energization based on an instruction from a user or another electronic device, and may be provided with or without a power source. The SMA wires 12, which are energized by an operation of the energization member 3, reach the predetermined temperature T or higher by heating through energization.

With a focus on each of the low-rigidity portions 11 included in the main body unit 2, it is preferable that the energization member 3 collectively switch presence or absence of energization to all the SMA wires 12 included in each of the low-rigidity portions 11. For example, in this embodiment, it is preferable that the energization member 3 collectively switch presence or absence of energization to the five SMA wires 12 included in each of the low-rigidity portions 11.

Note that the energization member 3 may only have a configuration of collectively switching energization to the SMA wires 12 included in all the low-rigidity portions 11 among the low-rigidity portions 11, or may have a configuration of switching energization to the SMA wires 12 included in some low-rigidity portions 11 selected among the low-rigidity portions 11.

In this illustrated embodiment, for example, the energization member 3 is electrically connected to the plurality of high-rigidity portions 10, and is electrically connected to the SMA wires 12 via the plurality of high-rigidity portions 10. The energization member 3 can change the interval where the SMA wires 12 are energized.

According to the rigidity variable device 1 having the configuration described above, when the plurality of SMA wires 12 are not energized, the temperature of the SMA wires 12 are less than the predetermined temperature T, and the elastic modulus of the SMA wires 12 are in a low state. According to the rigidity variable device 1, when the plurality of SMA wires 12 are energized, the temperature of the SMA wires 12 are equal to or higher than the predetermined temperature T, and the elastic modulus of the SMA wires 12 are in a high state.

The high-rigidity portions 10 and the low-rigidity portions 11 are alternately coupled along the longitudinal axis L in the rigidity variable device 1 in this embodiment, and only the rigidity of the low-rigidity portions 11 changes.

Since the high-rigidity portions 10 are metal columnar members, the high-rigidity portions 10 function as a rigid body even when a force in a direction of bending the longitudinal axis L of the main body unit 2 is inputted.

The low-rigidity portions 11 are made up of the plurality of SMA wires 12. Therefore, even if the elastic modulus of the SMA wires 12 is high, the low-rigidity portions 11 is elastically deformed in the bending direction when a force is applied in the direction of bending the longitudinal axis L of the main body unit 2. Consequently, in the rigidity variable device 1 in this embodiment, the rigidity of the main body unit 2 is changed in response to switching of presence or absence of energization to the plurality of SMA wires 12.

Since the main body unit 2 has the configuration where the plurality of high-rigidity portions 10 are connected by the plurality of SMA wires 12, the entire rigidity of the main body unit 2 is higher than the rigidity of a bundle of the same number of SMA wires having the same length as the main body unit 2.

Here, the low-rigidity portions 11 in this embodiment include the plurality of SMA wires 12 that intervene between the adjacent pair of high-rigidity portions 10, separately in directions orthogonal to the longitudinal axis L. In other words, the SMA wires 12 form beams having opposite ends fixed to the adjacent pair of high-rigidity portions 10. The low-rigidity portions 11 having such a configuration have a high rigidity, because the beams are apart from each other in the direction orthogonal to the longitudinal axis L even if each of the beams (SMA wires 12), which couple the adjacent pair of high-rigidity portions 10, is narrow.

Consequently, the rigidity variable device 1 in this embodiment can achieve a high rigidity when the SMA wires 12 are heated through energization to the predetermined temperature T or higher to increase the rigidity of the main body unit 2.

Since the low-rigidity portions 11 in this embodiment include the plurality of SMA wires 12 having a narrow diameter, the bending stress at a predetermined angle of the SMA wires 12 is smaller than, for example, the stress in a case where a single shape-memory alloy having a large diameter intervenes between the pair of high-rigidity portions 10. Accordingly, the angle of being elastically deformable in the bending direction is large. In other words, even when the main body unit 2 is deformed in the bending direction with a large curvature, the low-rigidity portions 11 are resistant to occurrence of permanent deformation and breakage.

In this embodiment, the plurality of SMA wires 12 constituting the low-rigidity portions 11 are linear members and have a low thermal capacity. Accordingly, a time period required to cool the SMA wires 12 from a state at the temperature equal to or higher than the predetermined temperature T to a state at the temperature lower than the predetermined temperature is very short. Consequently, the rigidity variable device 1 in this embodiment can perform switching from a state where the rigidity of the main body unit 2 is increased to a state where the rigidity is reduced in a short time period.

As described above, the rigidity variable device 1 in this embodiment can achieve a high rigidity when the rigidity of the main body unit 2 is increased, and achieve reduction in rigidity in a short time period in a compatible manner

Since the rigidity variable device 1 in this embodiment includes the plurality of SMA wires 12 having a small diameter, the power required for heating is smaller than the power in a case where a single shape-memory alloy intervenes between the pair of high-rigidity portions 10, for example. Accordingly, energization to the SMA wires 12 facilitates heating of the SMA wires 12 to the predetermined temperature T or higher. Consequently, the need for the heater for heating the SMA wires 12 can be negated.

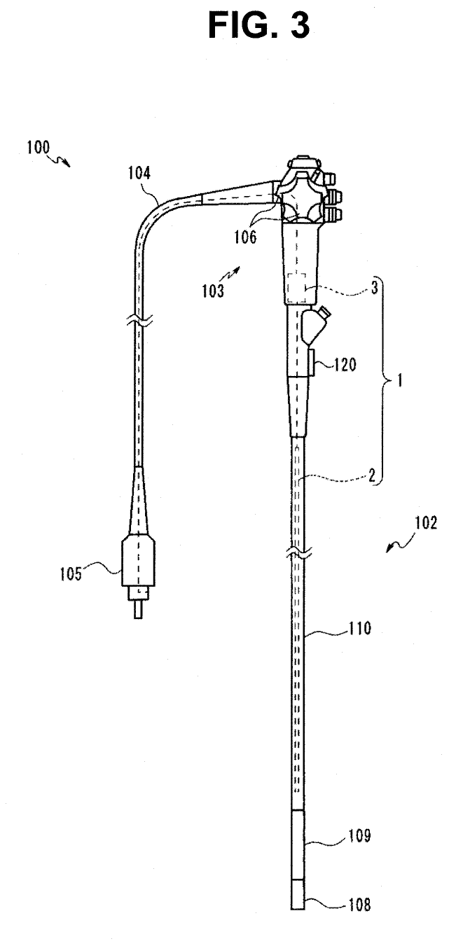

FIG. 3 shows an endoscope 100 that includes the rigidity variable device 1. The endoscope 100 includes an insertion portion 102 that can be introduced into a subject, such as a human body, and has an elongated shape and flexibility. The insertion portion 102 has a configuration for observing the inside of the subject. Note that the subject into which the insertion portion 102 of the endoscope 100 is introduced is not limited to a human body but may be another living body, or an artificial object, such as a machine or a building.

The endoscope 100 in this embodiment mainly includes the insertion portion 102, an operation portion 103 positioned at a proximal end of the insertion portion 102, and a universal cord 104 that extends from the operation portion 103.

The insertion portion 102 includes: a distal end portion 108 disposed at a distal end; a bending portion 109 that is disposed on a proximal end side of the distal end portion 108 and can be freely bent; and a flexible tube portion 110 that connects the proximal end of the bending portion 109 and the distal end of the operation portion 103 and has flexibility; and these components are continuously configured.

A configuration or the like for observing the inside of the subject is arranged at the distal end portion 108. For example, an image pickup unit that includes an objective lens and an image pickup device, and is for optically observing the inside of a subject, is arranged at the distal end portion 108. Although not shown, an illumination light emission unit that emits light with which the subject of the image pickup unit is to be illuminated, is also provided at the distal end portion 108. Note that an ultrasound transducer for acoustically observing the inside of the subject using ultrasound may be provided at the distal end portion 108.

The main body unit 2 of the rigidity variable device 1 is inserted in at least one of the bending portion 109 and the flexible tube portion 110, which can be subjected to bending deformation in the insertion portion 102. In this illustrated embodiment, for example, the main body unit 2 is arranged in the flexible tube portion 110.

The operation portion 103 arranged at the proximal end of the insertion portion 102 is provided with an angle operation knob 106 for operating the bending of the bending portion 109. The proximal end portion of the universal cord 104 is provided with an endoscope connector 105 configured to be connectable to an external apparatus (not shown). The external apparatus, to which the endoscope connector 105 is connected, includes a camera control unit that controls the image pickup unit provided in the distal end portion 108.

The operation portion 103 is provided with the energization member 3 of the rigidity variable device 1, and a rigidity changing switch 120 for controlling the energization member 3. The rigidity changing switch 120 controls an operation of switching presence or absence of energization to the SMA wires 12 through the energization member 3.

The energization member 3 is arranged in the operation portion 103. The energization member 3 is electrically connected to an electric contact provided at the endoscope connector 105 through an electric cable inserted in the universal cord 104. The power for heating the SMA wires 12 of the rigidity variable device 1 through energization is supplied from the external apparatus, to which the endoscope connector 105 is connected. Note that the endoscope 100 may include a battery that supplies the power for heating the SMA wires 12 of the rigidity variable device 1 through energization.

The endoscope 100 that has the configuration described above can change the rigidity of the elongated insertion portion 102 having flexibility, in response to the operation of the rigidity changing switch 120 by the user.

As described above, the rigidity variable device 1 in this embodiment can achieve a high rigidity when the rigidity of the main body unit 2 is increased, and reduction in rigidity in a short time period in a compatible manner Accordingly, the endoscope 100 can increase the variable range of the rigidity of the insertion portion 102, and reduce the time period required to change the rigidity in a compatible manner

The present invention is not limited to the embodiment described above, and can appropriately be changed in a range that is not against the gist or spirit of the invention understood from the claims and the entire description. Rigidity variable devices and endoscopes subjected to such change are also encompassed by the technical scope of the present invention.

What is claimed is:

1. A rigidity variable device, comprising:
an elongated main body unit;
a plurality of high-rigidity portions arranged with gaps along an longitudinal axis of the main body unit in the main body unit, the plurality of high-rigidity portions being made of metal; and
a low-rigidity portion including a plurality of shape-memory alloy wires that intervene between high-rigidity portions adjacent to each other among the high-rigidity portions, and are apart in a direction orthogonal to the longitudinal axis in the main body unit.

2. The rigidity variable device according to claim 1, further comprising
an energization member capable of switching presence or absence of energization to the plurality of shape-memory alloy wires,
wherein the shape-memory alloy wires are heated through the energization.

3. The rigidity variable device according to claim 1, wherein the number of high-rigidity portions is equal to or greater than three, and the number of low-rigidity portions is equal to or greater than two.

4. The rigidity variable device according to claim 3, wherein a plurality of the low-rigidity portions share identical shape-memory alloy wires of the plurality of shape-memory alloy wires.

5. The rigidity variable device according to claim 2, wherein the energization member is electrically connected to the plurality of high-rigidity portions, and is electrically connected to the shape-memory alloy wires via the plurality of high-rigidity portions.

6. An endoscope, comprising:
an insertion portion configured to be inserted into a subject; and
the rigidity variable device according to claim 1.
